(19) United States
(12) Patent Application Publication
(10) Pub. No.: US 2026/0317707 A1
(43) Pub. Date: Oct. 15, 2026

(54) METHOD FOR PRODUCING KERATINOCYTES AND KERATINOCYTES

(71) Applicant: Akihiro UMEZAWA, Tokyo (JP)

(72) Inventors: Akihiro UMEZAWA, Tokyo (JP); Ryo TAKAGI, Tokyo (JP)

(21) Appl. No.: 18/998,782

(22) PCT Filed: Jul. 18, 2023
(86) PCT No.: PCT/JP2023/026201

§ 371 Date: Jan. 27, 2025

(30) Foreign Application Priority Data
Jul. 29, 2022 (JP) 2022-121932

(51) Int. Cl. C12N 5/071; C12N 5/00

(52) U.S. Cl. CPC C12N 5/0629; C12N 5/0081; C12N 2501/727; C12N 2502/00; C12N 2506/45; C12N 2533/52; C12N 2533/90

(57) ABSTRACT

A method for producing keratinocytes includes a separation step of subjecting a cell population including keratinocytes induced from pluripotent stem cells to trypsinization so as to separate the keratinocytes, the separation step being carried out a plurality of times. Provided is a technique for producing a cell population of keratinocytes derived from pluripotent stem cells, the cell population including keratinocytes having high proliferation potency.

### TECHNICAL FIELD

The present invention relates to a method for producing keratinocytes, a medium kit for serially culturing keratinocytes, and keratinocytes.

### BACKGROUND ART

An attempt has recently been made to induce differentiation of pluripotent stem cells such as ES cells and iPS cells and produce various types of tissues and cells. For example, Non-patent Literatures 1 and 2 disclose techniques for inducing keratinocytes from pluripotent stem cells.

### CITATION LIST

### Non-patent Literature

### [Non-patent Literature 1]

Res Ther 2014; 5:60-70.

### [Non-Patent Literature 2]

Proc Natl Acad SciUSA 2006; 103:1792-7.

### SUMMARY OF INVENTION

### Technical Problem

However, the keratinocytes produced by the techniques disclosed in Non-patent Literatures 1 and 2 have lower proliferation potency and a shorter life as compared with those obtained by culturing human primary cells. Further, in the techniques disclosed in Non-patent Literatures 1 and 2, a method of introducing immortalizing genes is used, and resulting keratinocytes are not suitable for transplantation applications. Thus, a technique for producing, from pluripotent stem cells, keratinocytes that are suitable for transplantation applications has not been established.

An aspect of the present invention has been made in view of the foregoing problems, and an object thereof is to provide a technique for producing a cell population of keratinocytes derived from pluripotent stem cells, the cell population including keratinocytes having high proliferation potency.

### Solution to Problem

In order to attain the object, a method for producing keratinocytes in accordance with an aspect of the present invention includes a separation step of subjecting a cell population including keratinocytes induced from pluripotent stem cells trypsinization so as to separate the keratinocytes, the separation step being carried out a plurality of times.

A method for producing keratinocytes in accordance with another aspect of the present invention includes a culture step of serially culturing, in the presence of feeder cells, an Rho kinase inhibitor, and an extracellular matrix, keratinocytes induced from pluripotent stem cells.

A method for producing keratinocytes in accordance with a further aspect of the present invention includes a separation step of subjecting a cell population including keratinocytes induced from pluripotent stem cells to trypsinization so as to separate the keratinocytes, the separation step being carried out a plurality of times, the separated keratinocytes being serially cultured in the presence of feeder cells, an Rho kinase inhibitor, and an extracellular matrix.

A medium kit for serially culturing keratinocytes induced from pluripotent stem cells in accordance with an aspect of the present invention includes feeder cells, an Rho kinase inhibitor, and an extracellular matrix.

Keratinocytes in accordance with an aspect of the present invention are induced from pluripotent stem cells and have a cell population doubling level (PDL) of more than 15.

### Advantageous Effects of Invention

An aspect of the present invention makes it possible to provide a technique for producing a cell population of keratinocytes derived from pluripotent stem cells, the cell population including keratinocytes having high proliferation potency.

### BRIEF DESCRIPTION OF DRAWINGS

FIG. 1 is a view illustrating a trypsinization process of a method for producing keratinocytes in accordance with an aspect of the present invention.

FIG. 2 is a view illustrating cellular morphologies of control and trypsinization groups of hESC-derived keratinocytes in Examples.

FIG. 3 is a view illustrating a morphology of cells collected by first trypsinization in Examples.

FIG. 4 is a view illustrating a colony formation effect of hESC-derived keratinocytes in Examples.

FIG. 5 is a view illustrating morphologies of hESC-derived keratinocytes cultured under a serial culture condition and a fibroblast culture condition in Examples.

FIG. 6 is a view illustrating a result of gene expression analysis of hESC-derived keratinocytes in Examples.

FIG. 7 is a view illustrating a result of air-lifting culture of hESC-derived keratinocytes in Examples.

FIG. 8 is a view illustrating expression of markers in epidermal equivalents derived from hESC-derived keratinocytes in Examples.

FIG. 9 is a view illustrating a result of serial culture of hESC-derived keratinocytes in Examples.

FIG. 10 is a view illustrating a proliferation curve of hESC-derived keratinocytes during serial culture in Examples.

FIG. 11 is a view illustrating doubling times of the hESC-derived keratinocytes during serial culture in Examples.

FIG. 12 is a view illustrating cellular morphologies of hESC-derived keratinocytes cultured under five culture conditions in Examples.

FIG. 13 is a view illustrating cellular densities of the hESC-derived keratinocytes cultured under the five culture conditions in Examples.

FIG. 14 is a view illustrating doubling times of hESC-derived keratinocytes cultured under culture conditions 1 and 2 in Examples.

### DESCRIPTION OF EMBODIMENTS

### [Method for Producing Keratinocytes]

A method for producing keratinocytes in accordance with an aspect of the present invention includes a separation step of subjecting a cell population including keratinocytes induced from pluripotent stem cells to trypsinization so as to separate the keratinocytes, the separation step being carried out a plurality of times.

In the present specification, “pluripotent stem cells” need only be undifferentiated cells that have not differentiated and that have at least the capability of differentiating into keratinocytes. Keratinocytes are cells of which the epidermis is mainly composed, are also referred to as epidermal keratinized cells, and have proliferation potency and differentiation potency. The pluripotent stem cells may be human pluripotent stem cells. Examples of the pluripotent stem cells include embryonic stem cells (ES cells) isolated from early embryos, embryonic germ cells (EG cells) isolated from primordial germ cells during the fetal period, germline stem cells (GS cells) isolated from testes immediately after birth, stem cells derived from bone marrow such as iliac bone marrow and jaw bone marrow, mesenchymal stem cells such as adipose tissue-derived stem cells, and somatic cell-derived induced pluripotent stem cells which have pluripotency similar to that of ES cells and which are obtained by inducing dedifferentiation of subject's own somatic cells by introduction of a plurality of genes into somatic cells such as skin cells (or induced pluripotent stem cells (iPS cells)).

### (Separation Step)

In the separation step, the keratinocytes are separated from the cell population obtained in the process of inducing differentiation of the pluripotent stem cells. The cell population obtained in the process of inducing differentiation of the pluripotent stem cells can include not only the keratinocytes but also a number of other cell types and can be a miscellaneous cell population. Such a cell population may be an object obtained in the process of inducing differentiation of pluripotent stem cells into keratinocytes, or may be an object obtained in the process of inducing differentiation of pluripotent stem cells into somatic cells such as hepatocytes and muscle cells.

The cell population including keratinocytes induced from pluripotent stem cells can be obtained by a differentiation induction method conventionally known in the art. The method for producing keratinocytes in accordance with an aspect of the present invention may include a differentiation step of inducing differentiation of pluripotent stem cells to obtain a cell population including keratinocytes.

In an example, the cell population including keratinocytes induced from pluripotent stem cells may be a cell population obtained in inducing differentiation of hepatocytes and/or hepatic progenitor cells from pluripotent stem cells. Examples of a method for obtaining such a cell population include a method for inducing differentiation of hepatocytes and/or hepatic progenitor cells via formation of embryoid bodies from pluripotent stem cells (see also International Publication WO2019/131938, etc.). Differentiation of hepatocytes and/or hepatic progenitor cells is preferably induced by carrying out culture with a culture medium supplemented with a hepatic differentiation inducing factor. Examples of the hepatic differentiation inducing factor include: cytokines/cell growth factors such as hepatocyte growth factors (HGFs), Wnt, and R-spondin 1; Rho kinase inhibitors such as Y27632; extracellular matrices of laminin (or a fragment thereof), collagen, fibronectin, Matrigel, and the like; MAPK inhibitors; and the like. These may be used alone or in combination of two or more types.

The cell population including keratinocytes is preferably induced by adhesive culture. For example, adhesive culture is carried out with use of a vessel coated with any of the extracellular matrices as listed above and/or by co-culture with feeder cells. A selection pressure may be applied by carrying out culture with a medium supplied with an antibiotic(s). Examples of the antibiotic include penicillin, streptomycin, and amphotericin B. These may be used alone or in combination of two or more types.

In the separation step, the cell population including keratinocytes induced from pluripotent stem cells is subjected to trypsinization. The keratinocytes are separated from the cell population by carrying out, a plurality of times, the separation step of carrying out such trypsinization. The trypsinization is carried out under a condition in which the keratinocytes can be separated from the cell population. The trypsinization is, for example, such that the keratinocytes are separated from the cell population by detaching the cell population, which has adhered to a vessel for culturing cells, such as a culture vessel, from the vessel. For example, a trypsin-EDTA solution is used in the trypsinization in the separation step.

In the separation step, the keratinocytes are separated by a difference in adhesion of cells after the trypsinization. The keratinocytes have robust intercellular adhesion and robust capability of adhesion to an extracellular matrix. It is therefore assumed that the keratinocytes induced from pluripotent stem cells also have higher intercellular adhesion and higher capability of adhesion to an extracellular matrix than other cells. In the separation step, for example, the keratinocytes are separated from the cell population by carrying out trypsinization so as to detach, from a vessel, cells that are among the cell population having adhered to the vessel and that have lower adhesion than the keratinocytes, and remove the cells. The keratinocytes separated from the cell population may be detached from the vessel by further carrying out trypsinization.

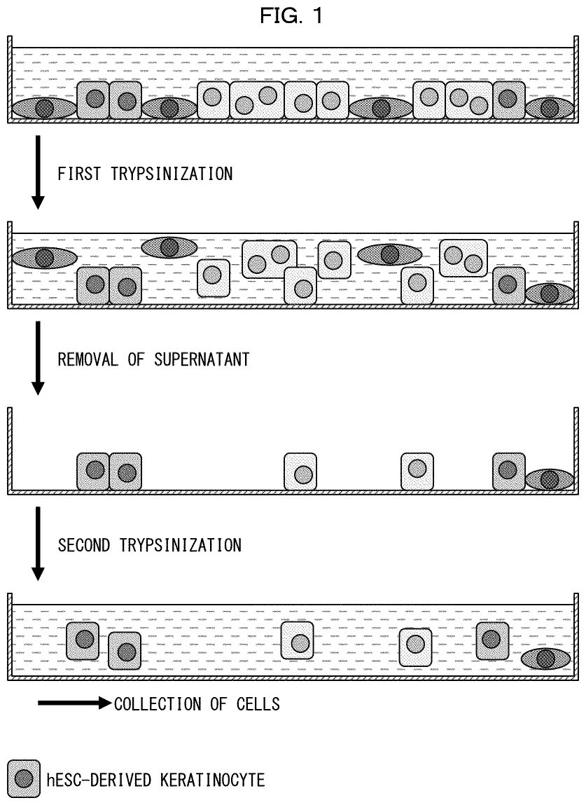

In the separation step, the keratinocytes are separated by (i) subjecting the cell population including the keratinocytes, the cell population having adhered to a vessel, to first trypsinization for detaching, from the vessel, cells different from the keratinocytes, and (ii) after the first trypsinization, subjecting the cell population having adhered to the vessel to second trypsinization for detaching the keratinocytes from the vessel. In the separation step, as illustrated in FIG. 1, the keratinocytes are separated from the cell population by the trypsinization carried out a plurality of times. FIG. 1 is a view illustrating a trypsinization process of a method for producing keratinocytes in accordance with an aspect of the present invention. Note that some of the keratinocytes can be detached in the separation step. Further, in a case where not all the cells different from the keratinocytes are removed in the separation step carried out once, purity of the keratinocytes can be increased by using cells subcultured after the separation step to further carry out the separation step a plurality of times.

As illustrated in FIG. 1, in the separation step, first, a vessel to which the cell population including keratinocytes induced from pluripotent stem cells having adhered is supplemented with trypsin so as to carry out the first trypsinization. In the first trypsinization, the cell population having adhered to the vessel is incubated in trypsin.

The first trypsinization is trypsinization carried out, with respect to the cell population including keratinocytes, the cell population having adhered to the vessel, under a treatment condition that cells different from the keratinocytes are detached from the vessel. By the first trypsinization, the cells different from the keratinocytes are detached from the vessel and float in a supernatant, and the keratinocytes remain adhering to the vessel. The cells that detach from the vessel by the first trypsinization can be cells having lower adhesion than the keratinocytes.

A trypsin concentration in the first trypsinization need only be in a range in which the cells different from the keratinocytes are detached and in which the keratinocytes are not detached in a first treatment step. A solution that is used in the first trypsinization may be a commonly used solution of 0.25 w/v % trypsin and 1 mmol/L EDTA. Further, a temperature of trypsin in the first trypsinization need only be in a temperature range in which the cells different from the keratinocytes are detached and in which the keratinocytes are not detached. The temperature is preferably room temperature or higher, and more preferably 37° C. However, it is desirable to note that a change(s) in concentration and/or temperature of a trypsin-EDTA solution change(s) enzyme activity, so that an appropriate treatment time is changed.

The cell population including the keratinocytes is exposed to trypsin for not less than one minute and not more than three minutes in the first trypsinization. In the first trypsinization, a time for which the cell population is exposed to trypsin need only be in a range in which the cells different from the keratinocytes are detached and in which the keratinocytes are not detached. The time is preferably three minutes. For example, in a case where the temperature is 37° C., exposing the cell population to the solution of 0.25 w/v % trypsin and 1 mmol/L EDTA for three minutes detaches the cells different from the keratinocytes from the vessel, but most of the keratinocytes remain adhering to the vessel.

Note that some of the keratinocytes having adhered to the inside of the vessel can be detached by the first trypsinization. In this case, keratinocytes remaining adhering to the inside of the vessel are preferably more than keratinocytes floating in the supernatant inside the vessel after the first trypsinization. Further, the first trypsinization and subsequent removal of the supernatant may be repeated a plurality of times before the second trypsinization. This makes it possible to further reduce an amount of the cells different from the keratinocytes in the vessel and increase the purity of the keratinocytes.

Subsequently, the supernatant in the vessel after the first trypsinization is removed. In the supernatant after the first trypsinization, the cells detached from the vessel by the first trypsinization are floating. Removing the supernatant removes, from the vessel, the cells detached by the first trypsinization. Cells including keratinocytes not having been detached by the first trypsinization are adhering to the vessel from which the supernatant has been removed.

The second trypsinization is trypsinization carried out, with respect to the cell population having adhered to the vessel after the first trypsinization, under a treatment condition that the keratinocytes are detached from the vessel. The keratinocytes can be separated by collecting, by the second trypsinization, the keratinocytes detached from the vessel. In the second trypsinization, the cell population having adhered to the vessel after the first trypsinization is incubated in trypsin.

By the second trypsinization, the keratinocytes are detached from the vessel and float in the supernatant. Cells having higher adhesion than the keratinocytes may remain adhering to the vessel by the second trypsinization. Further, the supernatant in the vessel after the second trypsinization may include other cells together with the keratinocytes. In this case, the keratinocytes are preferably included in the supernatant in a larger amount than other cells.

A trypsin concentration in the second trypsinization need only be in a range in which the keratinocytes are detached. A solution that is used in the second trypsinization may be a commonly used solution of 0.25 w/v % trypsin and 1 mmol/L EDTA. Further, a temperature of trypsin in the second trypsinization need only be in a temperature range in which the keratinocytes are detached. The temperature is preferably room temperature or higher, and more preferably 37° C.

The cell population from which the cells detached by the first trypsinization have been removed is exposed to trypsin for not less than three minutes in the second trypsinization. In the second trypsinization, a time for which the keratinocytes are exposed to trypsin need only be in a range in which the keratinocytes are detached. The time is more preferably not less than three minutes, not less than six minutes, or not less than nine minutes. For example, in a case where the temperature is 37° C., the keratinocytes are detached from the vessel by exposing the cell population to the solution of 0.25 w/v % trypsin and 1 mmol/L EDTA for not less than three minutes and not more than seven minutes.

Note that, in the first trypsinization and the second trypsinization, the concentration and the temperature of trypsin may be shared, and only a trypsinization time for which the cell population is exposed to trypsin may be changed. In this case, the first trypsinization need only be shorter in treatment time than the second trypsinization.

The separation step allowing the keratinocytes to be separated from the cell population including keratinocytes induced from pluripotent stem cells makes it possible to achieve a high purity of resulting keratinocytes and obtain various keratinocytes. Thus, the resulting keratinocytes can include keratinocytes having low proliferation potency, but are also highly likely to include keratinocytes having high proliferation potency. In a case where various keratinocytes are thus obtained, it is possible to culture the keratinocytes so as to select keratinocytes having high proliferation potency.

A conventional purification method using a cloning ring extremely limits the number of cells that can be collected. This may make it impossible to collect keratinocytes having high proliferation potency. Further, using a cloning ring to separate a plurality of colonies requires cloning rings as many as the number of colonies to be separated. According to the method for producing keratinocytes in accordance with an aspect of the present invention, cells that have been induced to differentiate as keratinocytes can be collected from the entire culture dish. This makes it possible to easily and stably produce keratinocytes having high proliferation potency. In a conventional technique, keratinocytes induced from pluripotent stem cells have low proliferation potency. However, according to the method for producing keratinocytes in accordance with an aspect of the present invention, it is possible to stably produce keratinocytes having high proliferation potency. Thus, even keratinocytes induced from pluripotent stem cells can be serially cultured for a long term.

### (Culture Step)

A method for producing keratinocytes in accordance with another aspect of the present invention includes a culture step of serially culturing, in the presence of feeder cells, an Rho kinase inhibitor, and an extracellular matrix, keratinocytes induced from pluripotent stem cells. The keratinocytes in the culture step can be keratinocytes separated, by the foregoing separation step, from a cell population induced from pluripotent stem cells. That is, the present invention also encompasses, in its scope, a method for producing keratinocytes, including a separation step of subjecting a cell population including keratinocytes induced from pluripotent stem cells to trypsinization so as to separate the keratinocytes, the separation step being carried out a plurality of times, the separated keratinocytes being serially cultured in the presence of feeder cells, an Rho kinase inhibitor, and an extracellular matrix. Note that the foregoing separation step may be carried out during serial culture to purify the keratinocytes.

In the present specification, the term “culture” means that cells are grown in vitro. Culturing cells with use of a medium means culturing cells in contact with the medium, such as culturing cells in the medium or culturing cells on the medium. The medium may be a medium in gel or liquid form.

The medium used in the culture step need only be any medium that allows keratinocytes to be cultured, and can be a keratinocyte culture medium conventionally known in the art. Such a culture medium can be a commercially-available medium, and examples of the culture medium include a keratinocyte serum-free medium (DK-SFM). The keratinocyte culture medium may contain other known component(s) for culturing keratinocytes.

In the culture step, the keratinocytes in the keratinocyte culture medium are cultured in the presence of feeder cells, an Rho kinase inhibitor, and an extracellular matrix. For example, the keratinocytes are seeded onto a feeder layer including feeder cells and are co-cultured in a keratinocyte culture medium containing an Rho kinase inhibitor and an extracellular matrix. In the culture step, the extracellular matrix may be coated on a vessel for culturing keratinocytes.

In the culture step, serially culturing the keratinocytes in the presence of feeder cells, an Rho kinase inhibitor, and an extracellular matrix makes it possible to culture the keratinocytes that maintain their proliferation potency. This makes it possible to achieve long-term serial culture.

The feeder cells are cells that act as a basal layer for keratinocytes and that provide a secretor factor, an extracellular matrix, and a cell contact for maintaining keratinocytes. The feeder cells can be prepared by being inactivated by gamma irradiation or the like. The feeder cells can be feeder cells conventionally known in the art for culture of keratinocytes, and examples of the feeder cells include mouse embryonic fibroblasts. The feeder cells can be used as a feeder layer prepared in layers. The feeder cells need only be used in the feeder layer in an amount that is suitable for serial culture of keratinocytes and that is, for example, preferably not less than 2.0×10⁴ cells and not more than 2.5×10⁴ cells per 1 cm², and more preferably 2.5×10⁴ cells per 1 cm².

The Rho kinase inhibitor inhibits action of Rho kinase, which is intracellular kinase, and suppresses cell death. The Rho kinase inhibitor can be an Rho kinase inhibitor conventionally known in the art and is, for example, Y27632.

The Rho kinase inhibitor can be used by being added to a medium for culturing keratinocytes and need only be added in an amount suitable for serially culturing the keratinocytes. The Rho kinase inhibitor is added in an amount of, for example, 10 μmol/L.

The extracellular matrix serves as a scaffold for cell culture and is a protein that is involved in adhesion, migration, proliferation, etc. of cells. The extracellular matrix can be an extracellular matrix conventionally known in the art and is, for example, laminin or a fragment thereof. Laminin may be produced from laminin-expressing cells by a known method, or a commercially available product of laminin may be purchased.

The extracellular matrix can be used by being coated on a vessel for culturing keratinocytes, and need only be coated in an amount suitable for serially culturing keratinocytes. For example, an extracellular matrix coating amount is preferably such that not less than 0.1 mL of a 2.5 μg/mL laminin-511E8 fragment solution is coated per area of 1 cm² of the vessel to be coated.

A culture condition in the culture step need only be a keratinocyte culture condition conventionally known in the art. In the culture step, the keratinocytes are cultured at, for example, not lower than 30° C. and not higher than 40° C., and preferably 37° C. Further, in the culture step, the keratinocytes are cultured, for example, in the presence of not less than 3% and not more than 15% carbon dioxide, and preferably in the presence of 5% carbon dioxide. Furthermore, in the culture step, the keratinocytes are cultured in a humidified environment. The keratinocytes are cultured, for example, in an environment at a humidity of not less than 85%, and preferably in an environment at a humidity of not less than 95%.

Culturing, in the presence of feeder cells, an Rho kinase inhibitor, and an extracellular matrix, keratinocytes induced from pluripotent stem cells makes it possible to culture keratinocytes for a long term while maintaining their proliferation potency. In a conventional technique, keratinocytes induced from pluripotent stem cells have low proliferation potency. However, according to the method for producing keratinocytes in accordance with an aspect of the present invention, even keratinocytes induced from pluripotent stem cells can be serially cultured for a long term.

Further, keratinocytes produced by the method for producing keratinocytes in accordance with an aspect of the present invention and keratinocytes produced by another method can be distinguished from each other in that the keratinocytes produced by the method for producing keratinocytes in accordance with an aspect of the present invention have high proliferation potency. The keratinocytes produced by the method for producing keratinocytes in accordance with an aspect of the present invention can be distinguished from other keratinocytes by a difference in molecular marker and gene expression. The present invention also encompasses, in its scope, a molecular marker for distinguishing the keratinocytes produced by the method for producing keratinocytes in accordance with an aspect of the present invention from other keratinocytes.

[Medium Kit for Serially Culturing Keratinocytes Induced from Pluripotent Stem Cells]

A medium kit for serially culturing keratinocytes induced from pluripotent stem cells in accordance with an aspect of the present invention includes feeder cells, an Rho kinase inhibitor, and an extracellular matrix. The medium kit can be used together with a medium for culturing keratinocytes induced from pluripotent stem cells. This makes it possible to culture keratinocytes for a long term while maintaining their proliferation potency. The medium kit may further include a keratinocyte culture medium.

The feeder cells, the Rho kinase inhibitor, and the extracellular matrix each included in the medium kit are identical to those used in the culture step of the method for producing keratinocytes in accordance with an aspect of the present invention. Thus, a description of the culture step of the foregoing method for producing keratinocytes in accordance with an aspect of the present invention is referred to for details of, for example, types and contents of the feeder cells, the Rho kinase inhibitor, and the extracellular matrix each included in the medium kit, and a culture method using these.

### [Keratinocytes]

Keratinocytes in accordance with an aspect of the present invention are induced from pluripotent stem cells and have a cell population doubling level (PDL) of more than 15. Further, the keratinocytes in accordance with an aspect of the present invention have a cell population doubling time of not more than 60 hours. The keratinocytes in accordance with an aspect of the present invention have high proliferation potency, and thus their proliferation potency is maintained even in a case where the keratinocytes are serially cultured.

Note here that the PDL is determined by dividing the number of cells in the cell population at the end of culture by the number of cells in the cell population at the start of culture. The keratinocytes in accordance with an aspect of the present invention preferably have a PDL of not less than 16, not less than 20, not less than 30, not less than 40, or not less than 45.

A cell doubling time is intended to mean a time required for the number of cells included in the cell population to double. The keratinocytes in accordance with an aspect of the present invention preferably have a cell population doubling time of not more than 55 hours, not more than 50 hours, not more than 45 hours, not more than hours, or not more than 30 hours.

The keratinocytes in accordance with an aspect of the present invention can be produced by the method for producing keratinocytes in accordance with an aspect of the present invention. Further, the keratinocytes in accordance with an aspect of the present invention can be produced with use of the medium kit for serially culturing keratinocytes induced from pluripotent stem cells in accordance with an aspect of the present invention.

The present invention is not limited to the embodiments, but can be altered by a skilled person in the art within the scope of the claims. The present invention also encompasses, in its technical scope, any embodiment derived by combining technical means disclosed in differing embodiments.

### EXAMPLES

### [1. Ethical Statement]

All experiments with use of human cells and tissues were approved by the Institutional Review Board at the National Institute of Biomedical Innovation. Informed consent was obtained from parents of a patient. ESC lines were derived and cultured in full compliance with “the Guidelines for Derivation and Distribution of Human Embryonic Stem Cells (Notification of the Ministry of Education, Culture, Sports, Science, and Technology in Japan (MEXT), No. 156 of Aug. 21, 2009; Notification of MEXT, No. 86 of May 20, 2010)” and “the Guidelines for Utilization of Human Embryonic Stem Cells (Notification of MEXT, No. 157 of Aug. 21, 2009; Notification of MEXT, No. 87 of May 20, 2010)”.

[2. Culture and Differentiation of hESCs]

SEES-2, which is a human embryonic stem cell (hESC) line established in previous study of the inventors of the present invention, was routinely cultured in an ESC culture medium having a feeder layer derived from mouse embryonic fibroblasts (Reference document 1 listed later). The mouse embryonic fibroblasts for maintaining SEES-2 were isolated from ICR mouse fetuses at 12.5 d gestation, serially cultured twice, and gamma-irradiated at 30 Gy (Hitachi, MBR-1520 R-3) so as to prepare the feeder layer. ESC media were adjusted by supplementing KO-DMEM (Thermo Fisher Scientific) with 20% KO-TM serum (KO-SR; Thermo Fisher Scientific), 2 mmol/L Glutamax-I (Thermo Fisher Scientific), 0.1 mmol/L non-essential amino acids (NEAA; Thermo Fisher Scientific), 1 mmol/L sodium pyruvate (Thermo Fisher Scientific), and 50 ng/ml recombinant human full-length bFGF (Thermo Fisher Scientific).

In order to generate embryoid bodies (EBs), SEES-2 was dissociated into single cells with Accutase (Thermo Fisher Scientific) after being exposed to Y-27632 (Fujifilm), which is an Rho kinase inhibitor, and the single cells were seeded in an EB medium at a density of 5×10³ cells/well in 96-well plates. A medium adjusted by mixing, with KO-DMEM, 20% KO-SR, 2 mmol/L GlutaMAX-I, 0.1 mmol/L NEAA, 1 mmol/L pyruvate, and 1 mmol/L sodium pyruvate was used as the EB medium.

The EBs were transferred to 24-well plates coated with collagen type I, and cultured in an XF32 medium for 35 days. A KO-DMEM medium supplemented with 15% Knockout Serum Replacement XF CTS (Thermo Fisher Scientific), 2 mmol/L GlutaMAX-I, 0.1 mmol/L NEAA, 1 mmol/L sodium pyruvate, 50 mg/mL1-ascorbic acid 2-phosphate, 10 ng/ml heregulin-1b (R&D Systems, MN, USA), 200 ng/ml recombinant human IGF-1 (Sigma-Aldrich), and 20 ng/ml human bFGF was used as the XF32 medium.

Differentiated cells were further co-cultured with the feeder layer of the mouse embryonic fibroblasts and expanded, in a humidified environment at 5% CO₂ and 37° C., in an ESTEM-HE medium (GlycoTechnica, Kanagawa, Japan) containing Wnt3a and R-spondin 1 (Reference document 2 listed later). When the culture solution reached subconfluence, the cells were harvested with use of 0.25% trypsin and 1 mmol/L EDTA (Fujifilm Wako Pure Chemicals) and re-plated at a density of ¼ confluence on 100 mm culture dishes. Thereafter, media were changed every three days. The differentiated hESCs were serially cultured and cryopreserved at passage 4.

[3. Purification and Serial Culture of hESC-Derived Keratinocytes]

In order to prepare feeder layers, mouse fibroblasts derived from calvarium bone marrow were serially cultured in alpha-modified Eagle's minimum essential medium (α-MEM: Thermo Fisher Scientific) containing 10% FBS (Thermo Fisher Scientific), 100 unit/mL penicillin, 100 mg/mL streptomycin (Thermo Fisher Scientific), and 0.25 mg/mL amphotericin B (Bristol-Myers Squibb, NY, USA). The feeder layers were prepared in accordance with a method presented in previous study (Reference document 3 listed later). Briefly, the mouse fibroblasts treated with 10 mg/mL mitomycin (nacalai tesque, Kyoto, Japan) for 2 hours were seeded at 2.5×10⁴ cells/cm² onto culture dishes coated with LN-511-E8 (Nippi, Tokyo, Japan) in accordance with the manufacturer's protocols, so that the feeder layers were prepared.

The cryopreserved differentiated hESCs were seeded at a density of 2.9×10⁴ cells/cm² onto culture dishes coated with LN-511-E8 (laminin 511E8), which is an extracellular matrix, and cultured. A keratinocyte serum-free medium (DK-SFM; Thermo Fisher Scientific) supplemented with 10 mmol/mL Y-27632, 100 unit/mL penicillin, 100 mg/mL streptomycin, and 0.25 mg/mL amphotericin B was used as a medium. The hESCs were cultured in a humidified environment at 5% CO₂ and 37° C. by being co-cultured with the feeder layers. In order to separate hESC-derived keratinocytes and purify a cell population, first trypsinization and second trypsinization were carried out before serial culture at passage 5, 6, and 7.

As the first trypsinization, the differentiated hESCs were treated with trypsin-EDTA in an environment at 5% CO₂ and 37° C. for 3 minutes to 6 minutes. Supernatants in the first trypsinization were removed after incubation to remove cells contained in the supernatants and not exhibiting a keratinocyte-like cellular morphology. After the first trypsinization, cells exhibiting the keratinocyte-like cellular morphology were separated by newly using trypsin-EDTA to subject cells having adhered to the culture dishes to second trypsinization for 3 minutes to 7 minutes.

It was confirmed at passage 6 whether non-keratinocyte cells are proliferative in the differentiated hESCs. A cell suspension, which is the supernatants in the first trypsinization, was cultured in each of (i) a conventional fibroblast culture condition using an α-MEM medium containing 10% FBS, penicillin, and streptomycin and (ii) a serial culture condition using a feeder layer and a DK-SFM medium containing Y-27632 and LN-511-E8.

After passage 8, the hESC-derived keratinocytes were serially cultured without additional trypsinization until the proliferation rate decreased. At passage 13, in order to confirm the non-keratinocyte cells, the hESC-derived keratinocytes were also cultured in an α-MEM medium containing 10% FBS. Population doubling levels (PDL) and doubling times of the hESC-derived keratinocytes were calculated during serial culture. In order to confirm an effect of trypsinization in cell separation, cells differentiated from SEES-2 were serially cultured without trypsinization before serial culture, and these cells were used as a control group.

[4. Clonogenicity Analysis for hESC-Derived Keratinocytes]

hESC-derived keratinocytes at passage 7 (PDL3.7) were used as control and trypsinization groups and seeded at a density of 100 cells/well onto 6-well plates, and co-cultured with a mouse feeder layer in a keratinocyte culture medium (KCM) supplemented with 10 mmol/L Y-27632. The composition of KCM was in accordance with that used in previous study (Reference document 4 listed later). Briefly, KCM is obtained by mixing, at a ratio of 3:1, (i) Dulbecco's modified Eagle's medium (Sigma-Aldrich, MO, USA) and (ii) Ham's F-12 nutrient mixture (Thermo Fisher Scientific) supplemented with 100 unit/mL penicillin, 100 mg/mL streptomycin, 0.25 mg/mL amphotericin B, 5% FBS, 5 mg/mL insulin (Humulin; Eli Lilly, IN, USA), 10 ng/ml human recombinant epidermal growth factor (Higeta Shoyu, Chiba, Japan), 1 nmol/L cholera toxin (Fujifilm Wako Pure Chemicals), 2 nmol/L triiodothyronine (Fujifilm Wako Pure Chemicals), and 0.4 mg/mL hydrocortisone (Saxizon; Teva Takeda Pharma, Aichi, Japan).

After approximately 2 weeks of culture, the hESC-derived keratinocytes were stained with crystal violet, and percentages of colony formation efficiency were calculated by a method conventionally known in the art (Reference document 5 listed later). Colonies of hESC-derived keratinocytes were classified into the following three groups: keratinocyte colonies, non-stratified epithelial colonies, and fibroblast-like colonies.

[5. Gene Expression Analysis for hESC-Derived Keratinocytes]

Gene expression analysis for keratin 14 (KRT14), tumor protein p63 (TP63), involucrin (IVL), and filaggrin (FLG) was carried out with use of sequence data of primers in accordance with previous study (Reference document 6 listed later). Briefly, total RNA samples were prepared from hESC-derived keratinocytes at passage 5 and 9 with use of an RNeasy Plus kit (Qiagen, Hilden, German) and were used for cDNA synthesis by SuperScript III Reverse Transcriptase (Thermo Fisher Scientific).

mRNA expression was analyzed with use of Platinum SYBR Green qPCR SuperMix-UDG (Thermo Fisher Scientific) and Applied Biosystems

Quantstudio 12K Flex Real-Time PCR System (Thermo Fisher Scientific). Expression levels were normalized by expression of glyceraldehyde-3-phosphate dehydrogenase (GAPDH) and shown as a mean±standard deviation (SD) of percentages of GAPDH (% GAPDH).

Total RNA samples were prepared from postnatal human epidermal keratinocytes at passage 3 and used as positive controls for detecting gene expression. The keratinocytes were co-cultured for 8 days with a mouse feeder layer in KCM supplemented with 10 mmol/L Y-27632. The cultured keratinocytes were cultured for 3 days in KCM supplemented with Y-27632, so that terminal differentiation was induced.

### [6. Air-Lifting Culture Method]

hESC-derived keratinocytes at passage 16 (PDL31) were used for production of stratified epidermal equivalents by air-lifting culture in accordance with a method conventionally known in the art. Postnatal epidermal keratinocytes at passage 5 were used as control cells. Briefly, mouse feeder layers were seeded at a density of 2.5×10⁴ cells/cm² onto 6-well cell culture inserts (Corning, NY, USA) coated with LN-511-E8.

Onto the inserts, the hESC-derived keratinocytes were seeded at a density of 1×10⁴ cells/cm², and the postnatal epidermal keratinocytes were seeded at a density of 0.5×10⁴ cells/cm². These keratinocytes were cultured in KCM supplemented with 10 mmol/L Y-27632. After 7 days of culture, culture media in the culture inserts were discarded, and air-lifting culture was carried out in KCM without Y-27632. After 10 days of culture, the keratinocytes were collected, as stratified epidermal equivalents, from the culture inserts and fixed with a 20% formalin solution.

### [7. Histological Analysis for Epidermal Equivalents]

The fixed epidermal equivalents of cultured keratinocytes derived from hESCs and air-lifting cultured postnatal epidermis were processed into 5-mm-thick paraffin wax embedded sections in accordance with the usual method, and hematoxylin and cosin (HE) staining was carried out by a standard method. Expression of E-cadherin (CDH1), pan-keratins (KRTs), KRT14, keratin (KRT10), IVL, TP63, and vimentin (VIM) was analyzed by an immunohistological method. The embedded sections were treated with mouse monoclonal anti-CDH1 (1:100, 36/E-Cadherin, Becton Dickinson, NJ, USA), mouse monoclonal anti-KRTs (1:100 dilution, AE1/AE3, Thermo Fisher Scientific), mouse monoclonal anti-KRT14 (1:100, LL002, Abcam, Cambridge, UK), rabbit polyclonal anti-KRT10 (1:100, BioLegend, CA, USA), mouse monoclonal anti-IVL (1:1000, SY5, Sigma-Aldrich), mouse monoclonal anti-TP63 (1:100, 4A4, Abcam), and mouse monoclonal anti-VIM (1:100, Vim 3B4, Dako, Agilent Technologies, CA, USA) at room temperature for 90 minutes and were stained with peroxidase-conjugated secondary antibodies (Nichirei Bioscience, Tokyo, Japan) in accordance with the manufacturer's protocol. Human dermal and epidermal tissues were used for negative controls for immunohistological analysis.

### [8. Statistical Analysis]

Unpaired t-tests with Bonferroni correction were carried out for clonogenicities of control and trypsinization groups of keratinocytes, non-stratified epithelial cells, and fibroblast-like cells (n=6). Gene expression of hESC-derived keratinocytes at passage 5 and 9 and postnatal epidermal keratinocytes was analyzed by unpaired t-tests with Bonferroni correction (n=3). Densities of hESC-derived cells were measured in order to confirm effects of medium conditions, and analyzed by unpaired t-tests with Bonferroni correction (n=3). Doubling times of hESC-derived keratinocytes with or without Y-27632 were similarly analyzed by unpaired t-tests (n=3).

### [9. Results]

(Separation of hESC-Derived Keratinocytes)

Before serial culture, sensitivity to trypsin was used to separate keratinocytes from a hESC-derived differentiated cell population. That is, before serial culture, a population of cells differentiated from hESCs was treated with trypsin EDTA to remove supernatants, so that cells different from keratinocyte-like cells were removed. After trypsinization, cells having adhered to culture dishes were collected to be subjected to serial culture.

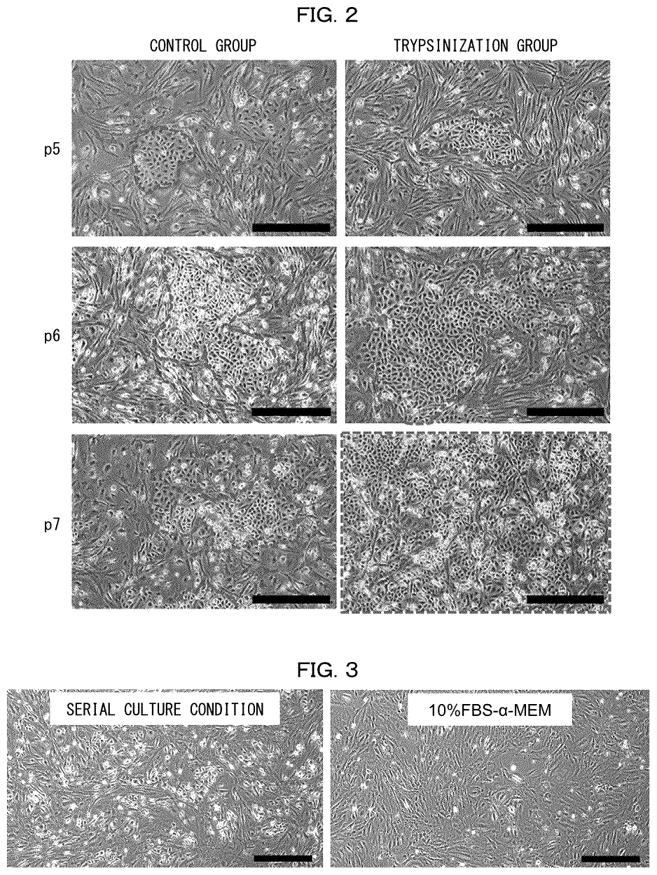

As illustrated in FIG. 2, in a trypsinization group, the proportion of keratinocytes further increased as serial culture progressed, as compared with a control group. FIG. 2 is a view illustrating cellular morphologies of control and trypsinization groups of hESC-derived keratinocytes at passage 5, 6, and 7. In FIG. 2, the drawings in the left column indicate the control group, and the drawings in the right column indicate the trypsinization group. In FIG. 2, the drawings in the upper row indicate passage 5 (p5), the drawings in the middle row indicate passage 6 (p6), and the drawings in the bottom row indicate passage 7 (p7). In FIG. 2, the keratinocytes in the differentiated hESC cell population are shown by dashed lines. Scale bars in FIG. 2 indicate 500 mm.

Cells collected by first trypsinization at passage 6 (PDL2.0) were cultured under a serial culture condition and by a conventional fibroblast culture method using α-MEM supplemented with FBS (FIG. 3). Since keratinocytes do not proliferate in a conventional fibroblast medium, proliferative cells observed in the α-MEM supplemented with FBS were classified as proliferative non-keratinocytes. FIG. 3 is a view illustrating a morphology of cells collected by the first trypsinization at passage 6 (PDL2.0). The cells were cultured under the serial culture condition (the drawing on the left in FIG. 3). The cells were also seeded onto normal culture dishes without a feeder layer and cultured in an α-MEM medium supplemented with 10% fetal bovine serum (FBS) (10% FBS-α-MEM, the drawing on the right in FIG. 3). Under this culture condition, non-keratinocyte cells were observed in 10% FBS-α-MEM. Scale bars in FIG. 3 indicate 500 mm.

These results show that additional trypsinization is useful for separating keratinocytes from other differentiated hESCs.

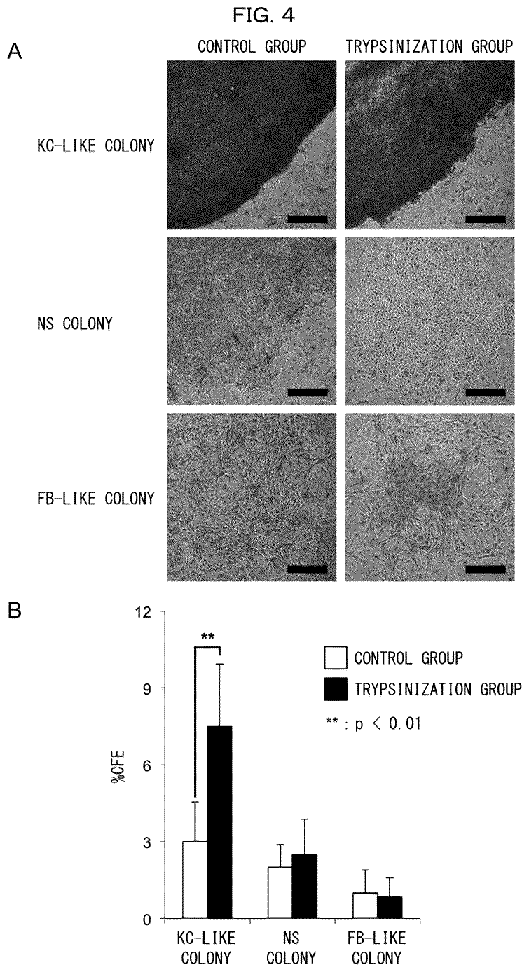

In order to confirm the effect of trypsinization, clonogenicities of the control and trypsinization groups were analyzed at passage 7. During culture, three types of colonies, which are classified as keratinocyte colonies, non-stratified epithelial colonies, and fibroblast-like colonies, were observed in both of the groups (A of FIG. 4). A colony formation effect of keratinocytes in the trypsinization group was significantly higher than in the control group (B of FIG. 4). Although fibroblast-like cells were not observed during serial culture of the trypsinization group at passage 7 (PDL3.7) (see FIG. 2), no such colonies were observed in both of the groups (A of FIG. 4).

FIG. 4 is a view illustrating a colony formation effect of hESC-derived keratinocytes. A of FIG. 4 is a view illustrating morphologies of hESC-derived keratinocytes in the control group (left column) and the trypsinization group (right column). Three types of colonies were observed in a microscopic image and classified as keratinocyte (KC), non-stratified epithelial (NS), and fibroblast (FB)-like colonies. Scale bars in FIG. 4 indicate 500 mm. B of FIG. 4 shows a percentage of a colony formation effect (% CFE) of hESC-derived keratinocytes in each of the control and trypsinization groups. Percentages of the colony formation effect were calculated for the respective three types of colonies and compared between the control and trypsinization groups. In B of FIG. 4, white bar graphs show results for the control group, and black bar graphs show results for the trypsinization group.

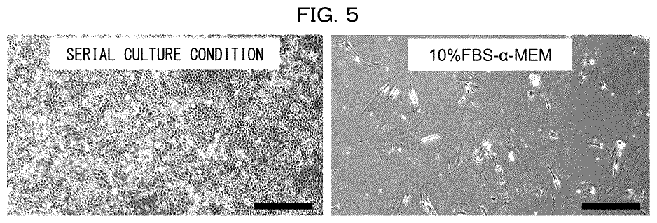

In order to confirm the presence of a proliferative non-keratinocyte cell population after further serial culture, normal culture dishes without a mouse feeder layer were used to culture hESC-derived keratinocytes at passage 13 (PDL23) in an α-MEM medium supplemented with 10% FBS. hESC-derived keratinocytes were merely observed under a serial culture condition, and no proliferative non-keratinocytes were observed in the α-MEM (FIG. 5). FIG. 5 illustrates morphologies of the hESC-derived keratinocyte at passage 13 (PDL23). The drawing on the left in FIG. 5 shows a result for a serial culture condition, and the drawing on the right in FIG. 5 shows a result for a fibroblast culture condition. The hESC-derived keratinocytes were also seeded onto an α-MEM medium supplemented with 10% FBS on normal culture dishes without a mouse feeder layer. Under this culture condition, proliferative non-keratinocyte cells were not observed in the hESC-derived keratinocytes. Scale bars in FIG. 5 indicate 500 mm.

These results show that trypsinization is useful for separation of keratinocytes from a differentiated hESC cell population. The serial culture condition did indeed reduce a proliferative property of non-keratinocyte cells in the hESC-derived keratinocytes.

(Gene Expression of hESC-Derived Keratinocytes)

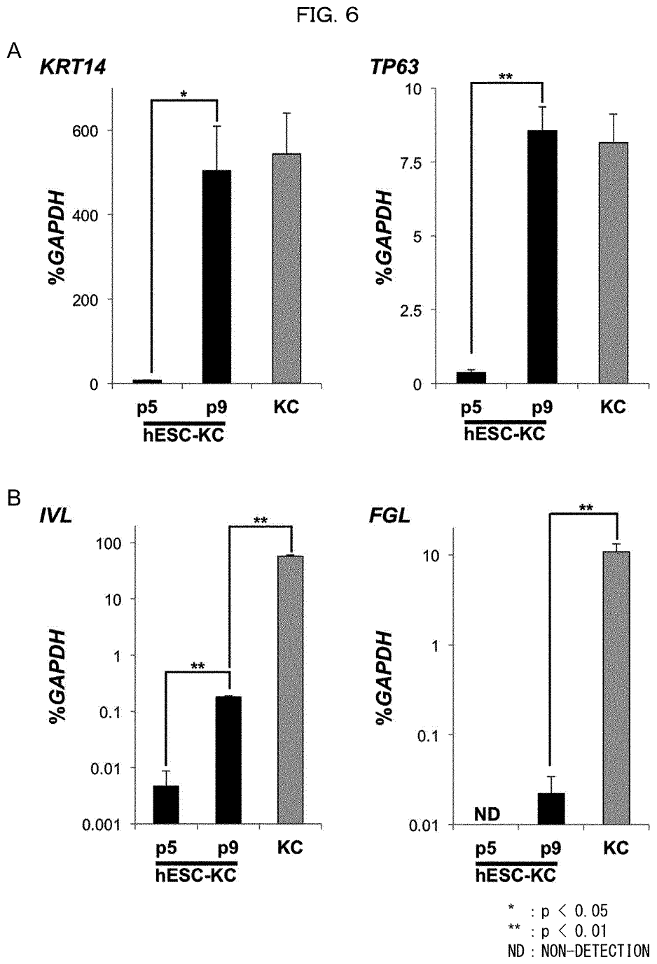

In order to demonstrate a clonogenicity of hESC-derived keratinocytes, gene expression analysis was carried out to confirm by qRT-PCR whether the hESC-derived keratinocytes contained keratinocyte progenitor cells. The gene expression analysis was carried out for expression of KRT14, which is known as an epithelial tissue basal cell marker, and TP63, which is a keratinocyte progenitor cell marker. hESC-derived keratinocytes at passage 9 exhibited significantly higher expression than those at passage 5 (A of FIG. 6).

FIG. 6 is a view illustrating a result of gene expression analysis of hESC-derived keratinocytes. Cultured human epidermal keratinocytes (KC) were used as positive controls for qRT-PCR. A of FIG. 6 shows results of marker gene expression of keratinocyte progenitor cells in hESC-derived keratinocytes at passage 5 and 9 (p5 and p9, respectively). Gene expression of each of keratin 14 (KRT14) and tumor protein p63 (TP63) is presented as a mean±SD of percentages of glyceraldehyde-3-phosphate dehydrogenase (% GAPDH). B of FIG. 6 shows keratinocyte terminal differentiation marker gene expression in the hESC-derived keratinocytes at passage 5 and 9. Gene expression of each of involucrin (IVL) and filaggrin (FLG) is presented as a mean±SD of % GAPDH. In B of FIG. 6, the Y-axis shows a logarithm.

Interestingly, there was no significant difference in gene expression of KRT14 and TP63 between the hESC-derived keratinocytes at passage 9 and cultured human epidermal keratinocytes at passage 3. Gene expression of keratinocyte terminal differentiation markers IVL and FGL in the hESC-derived keratinocytes at passage 9 was significantly lower than in the epidermal keratinocytes. These results of gene expression analysis show that keratinocyte progenitor cells are abundant in trypsinized and serially cultured hESC-derived keratinocytes and that cell terminal differentiation is suppressed under a serial culture condition using a serum-free low calcium ion medium and Y-27632.

(Stratified Epidermal Equivalent with hESC-Derived Keratinocytes)

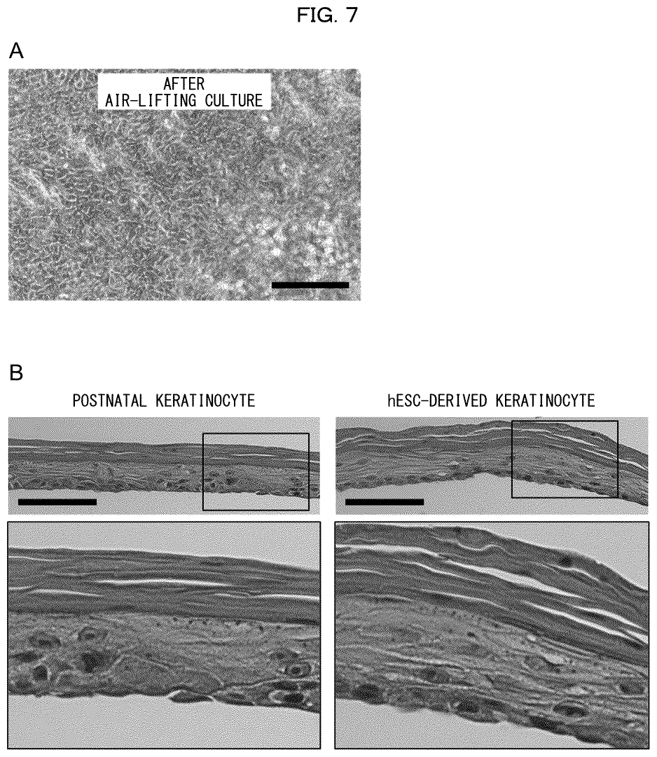

hESC-derived keratinocytes were successfully separated from the differentiated hESC cell population by additional trypsinization and serial culture. In order to confirm differentiation potency of the hESC-derived keratinocytes to terminally differentiate into stratified epidermal keratinocytes, the keratinocytes were seeded onto cell culture inserts for air-lifting culture. Histological analysis of air-lifting culture has shown that, as in the case of postnatal keratinocytes, hESC-derived keratinocytes formed stratified epidermal equivalents having cornified layers (B of FIG. 7). FIG. 7 is a view illustrating a result of air-lifting culture of hESC-derived keratinocytes. A of FIG. 7 illustrates a morphology of hESC-derived keratinocytes after air-lifting culture. B of FIG. 7 illustrates paraffin embedded sections of the equivalents, the paraffin embedded sections being stained with haematoxylin and eosin. Scale bars in FIG. 7 indicate 200 mm.

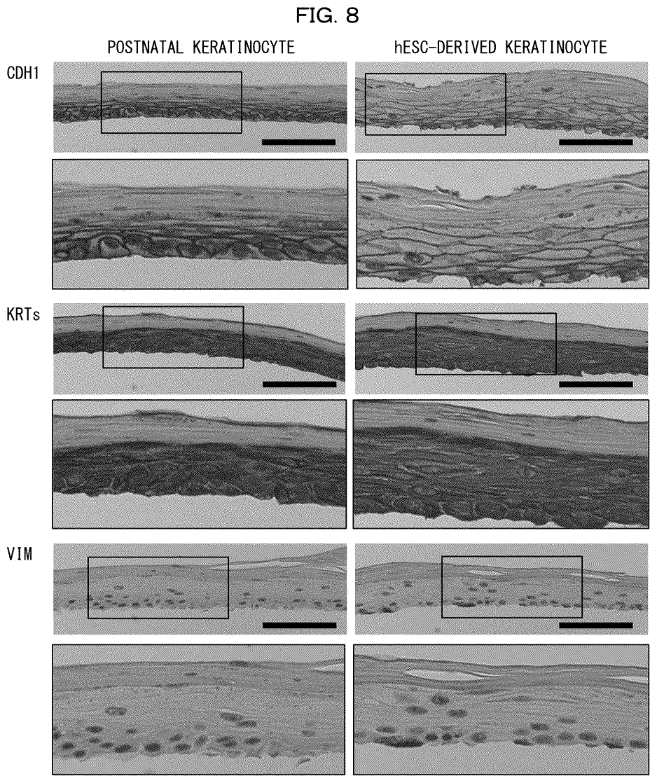

Epithelial cell markers CDH1 and KRTs were expressed in hESC-derived keratinocytes and epidermal tissues, and VIM, which is a mesenchymal cell marker, was not expressed (FIG. 8). Further, hESC- and epidermis-derived keratinocytes expressed not only the progenitor cell markers KRT14 and TP63 but also terminal differentiation markers KRT10 and IVL (FIG. 8). FIG. 8 is a view illustrating expression of markers in epidermal equivalents derived from hESC-derived keratinocytes. The epidermal equivalents were produced by air-lifting culture. In FIG. 8, the drawings in the left column illustrate postnatal human epidermal keratinocytes, and the drawings in the right column illustrate hESC-derived keratinocytes. The equivalents were analyzed by an immunohistological method to confirm expression of E-cadherin (CDH1), pan-keratins (KRTs), KRT14, keratin (KRT10), IVL, TP63, and vimentin (VIM). Scale bars in FIG. 8 indicate 200 mm.

Percentages of TP63 positive cells in the hESC-derived keratinocytes and the postnatal epidermal keratinocytes were 36.7±3.0 and 33.0±5.4, respectively (mean±SD). This shows that there is no significant difference between the two types of keratinocytes. There results show that differentiation potency of hESC-derived keratinocytes is maintained also during long-term serial culture under conditions of the present experiment.

(Effect of Y-27632 and LN-511-E8 on hESC-Derived Keratinocytes)

After separation by trypsinization, hESC-derived keratinocytes were serially cultured together with a mouse feeder layer on culture dishes coated with LN-511-E8 in DK-SFM supplemented with Y-27632.

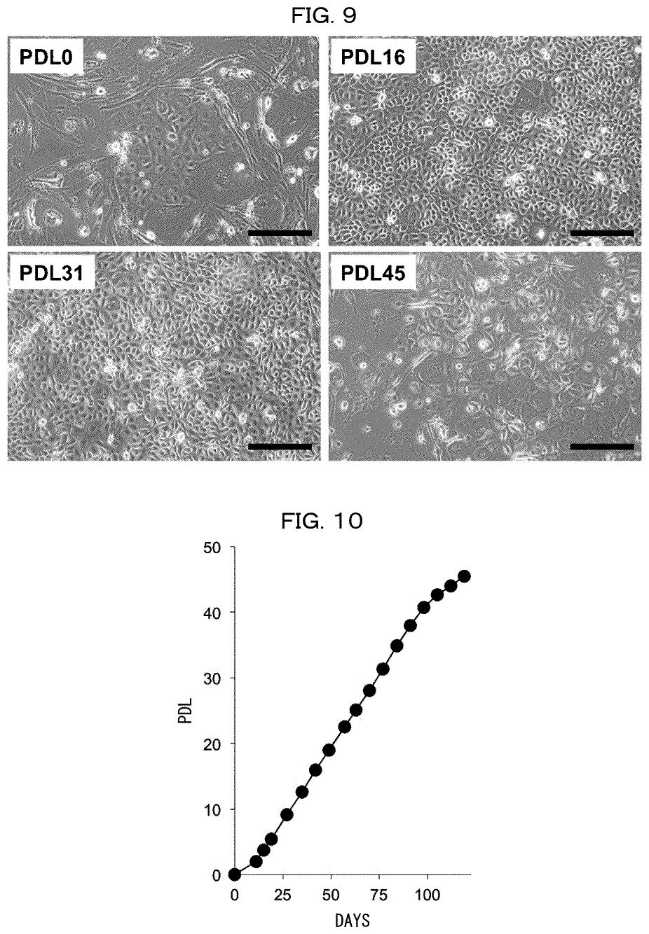

FIG. 9 shows a result of serial culture of hESC-derived keratinocytes and shows PDL of hESC-derived keratinocytes from passage 5 (PDLO) to passage 22 (PDL45). The PDL of the keratinocytes at passage 5 before separation by trypsinization was defined as 0. FIG. 9 illustrates morphologies of hESC-derived keratinocytes during serial culture at passage 5 (PDLO), passage 11 (PDL16), passage 16 (PDL31), and passage 22 (PDF45). Scale bars in FIG. 9 indicate 200 mm.

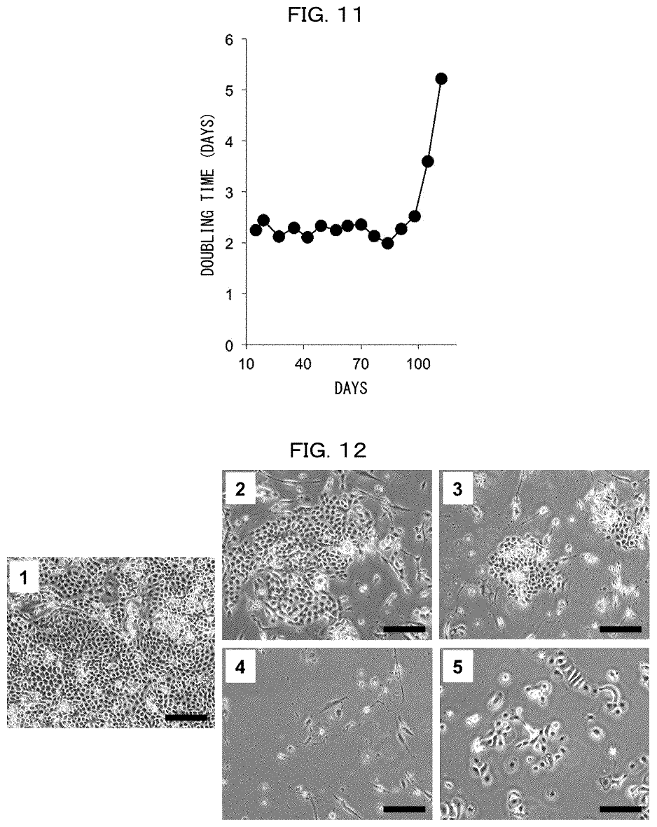

PDL from passage 5 (PDLO) to passage 22 (PDL45) and doubling times from passage 7 (PDL3.7) to passage 21 (PDL44) are calculated and shown in FIGS. 10 and 11. FIG. 10 shows a proliferation curve f the hESC-derived keratinocytes during serial culture, the proliferation curve being indicated by PDL from passage 5 (PDLO) to passage 22 (PDL45). FIG. 11 shows doubling times of the hESC-derived keratinocytes during serial culture from passage 7 (PDL3.7) to passage 21 (PDL44).

Subcultured hESC-derived keratinocytes exhibited a logarithmic growth phase up to passage 19 (PDL41) and had a doubling time of 2.3±0.14 days (mean±SD) during the growth phase. This result shows that Y-27632 and LN-511-E8 may be components essential for promotion of long-term culture.

### (Examination of Serial Culture Condition)

The necessity of components included in the serial culture condition was confirmed as shown below. hESC-derived keratinocytes were cultured under five culture conditions shown in Table 1 below.

TABLE 1

| Culture condition | 1 | 2 | 3 | 4 | 5 |
|---|---|---|---|---|---|
| LN511-E8 (2.5 μg/mL) | + | + | − | − | + |
| Y-27632 (10 μmol/L) | + | − | + | − | + |
| Feeder layer (2.5 × 10⁴/cm²) | + | + | + | + | − |
| Culture (days) | 7 | 12 | 12 | 12 | 12 |

A culture condition 1 is a serial culture condition using LN-511-E8, Y-27632, and the feeder layer. A culture condition 2 is a condition obtained by removing Y-27632 from the culture condition 1. A culture condition 3 is a condition obtained by removing LN-511-E8 from the culture condition 1. A culture condition 4 is a condition obtained by removing LN-511-E8 and Y-27632 from the culture condition 1. A culture condition 5 is a condition obtained by removing the feeder layer from the culture condition 1.

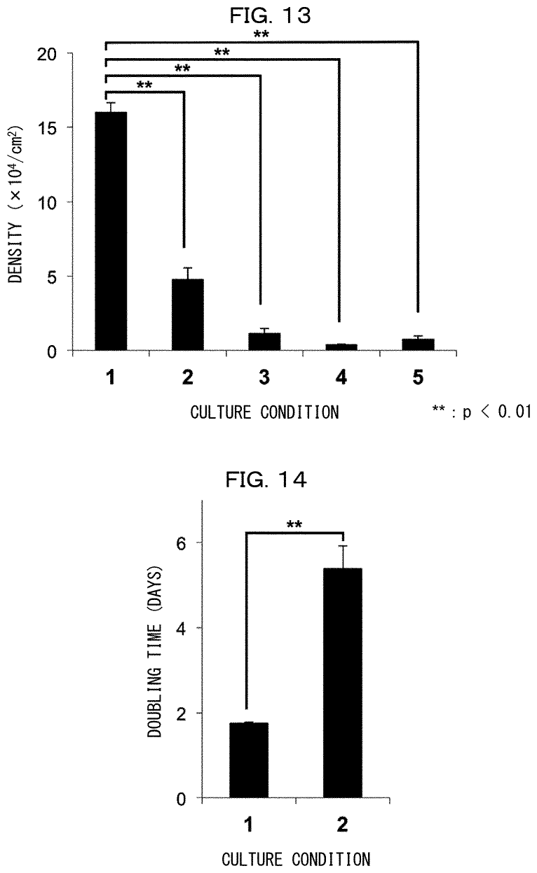

The necessity of LN-511-E8, Y-27632, and a feeder layer for maintenance of a cell proliferative property was confirmed by evaluating the five culture conditions for the hESC-derived keratinocytes for a cellular density after culture and a cell doubling time. FIG. 12 shows microscopic images of hESC-derived keratinocytes cultured under each of the culture conditions, and FIG. 13 shows cellular densities of the hESC-derived keratinocytes. FIG. 12 is a view illustrating cellular morphologies of hESC-derived keratinocytes cultured under five culture conditions. Scale bars in FIG. 12 indicate 200 mm. FIG. 13 illustrates cellular densities of the hESC-derived keratinocytes cultured under the five culture conditions.

As illustrated in FIGS. 12 and 13, the hESC-derived keratinocytes cultured under the culture condition 1 had a significantly higher cellular density than the hESC-derived keratinocytes cultured under the culture conditions 2 to 5.

FIG. 14 shows a result of comparison of doubling times between the culture condition 1 and the culture condition 2. FIG. 14 illustrates doubling times of the hESC-derived keratinocytes cultured under the culture conditions 1 and 2. As illustrated in FIG. 14, the hESC-derived keratinocytes cultured under the culture condition 1 had a significantly shorter doubling time than the hESC-derived keratinocytes cultured under the culture conditions 2 to 5.

These results have shown that the serial culture condition including LN-511-E8, Y-27632, and the feeder layer enables long-term serial culture of hESC-derived keratinocytes.

### (Examination of Trypsinization Condition)

Already purified hESC-derived keratinocytes were used to measure, for each trypsinization time, a percentage of cells detached. The hESC-derived keratinocytes were treated with a solution of 0.25 w/v % trypsin and 1 mmol/L EDTA at 37° C. A percentage of cells contained in a supernatant after treatment was measured to be 5.63±0.54% (n=3) in the case of a trypsinization time of 3 minutes, 11/1±0.82% (n=3) in the case of a trypsinization time of 6 minutes, and 60.9±8.76% (n=3) in the case of a trypsinization time of 9 minutes.

In contrast, in an aspect of the present invention, cells removed by the first trypsinization are considered to be composed mainly of fibroblasts due to their cellular morphology. Thus, hESC-derived fibroblasts were prepared to compare the number of cells collected by trypsinization with that of hESC-derived keratinocytes.

In a case where the hESC-derived fibroblasts were treated with a solution of 0.25 w/v % trypsin and 1 mmol/L EDTA at 37° C. for 3 minutes, all cells were observed to be detached from a culture surface. In contrast, it has been confirmed that, in a case where the hESC-derived fibroblasts were treated with a solution of 0.25 w/v % trypsin and 1 mmol/L EDTA at 37° C. for 1 minute, many cells remained adhering to culture dishes. The number of cells collected after trypsinization was measured to be 14.1±0.79 (×10⁵ cells/culture dish, n=3) in the case of a trypsinization time of 3 minutes and 5.3±0.7 (×10⁵ cells/culture dish) in the case of a trypsinization time of 1 minute. It has been confirmed that there is a significant difference in amount of collected cells by a difference in time of treatment with trypsin also for the hESC-derived fibroblasts (t-test, p<0.01).

### REFERENCE DOCUMENTS

For the reference documents mentioned in the present specification, see the following:
Reference document 1: Akutsu H, Machida M, Kanzaki S, Sugawara T, Ohkura T, Nakamura N, et al. Xenogeneic-free defined conditions for derivation and expansion of human embryonic stem cells with mesenchymal stem cells. Regen Ther 2015; 1:18-29
Reference document 2: Yachida S, Wood L D, Suzuki M, Takai E, Totoki Y, Kato M, et al. Genomic sequencing identifies ELF3 as a driver of ampullary carcinoma. Cancer Cell 2016; 29:229-40
Reference document 3: Takagi R, Yamato M, Kushida A, Nishida K, Okano T. Profiling of extracellular matrix and cadherin family gene expression in mouse feeder layer cells: type VI collagen is a candidate molecule inducing the colony formation of epithelial cells. Tissue Eng 2012; 18:2539-48 Reference document 4: Takagi R, Yamato M, Murakami D, Kondo M, Yang J, Ohki T, et al. Preparation of keratinocyte culture medium for the clinical applications of regenerative medicine. J Tissue Eng Regen Med 2011; 5: e63-73.
Reference document 5: Rheinwald J G, Green H. Serial cultivation of strains of human epidermal keratinocytes: the formation of keratinizing colonies from single cells. Cell 1975; 6:331-43.
Reference document 6: Kajiwara K, Tanemoto T, Wada S, Karibe J, Ihara N, Ikemoto Y, et al. Fetal therapy model of myelomeningocele with three-dimensional skin using amniotic fluid cell-derived induced pluripotent stem cells. Stem Cell Rep 2017; 8:1701-13.

### INDUSTRIAL APPLICABILITY

The present invention is applicable to a medical field.

1. A method for producing keratinocytes, comprising a separation step of subjecting a cell population including keratinocytes induced from pluripotent stem cells to trypsinization so as to separate the keratinocytes, the separation step being carried out a plurality of times.

2. The method as set forth in claim 1, wherein, in the separation step, the keratinocytes are separated by a difference in adhesion of cells after the trypsinization.

3. The method as set forth in claim 2, wherein
in the separation step, the keratinocytes are separated by
(i) subjecting the cell population including the keratinocytes, the cell population having adhered to a vessel, to first trypsinization for detaching, from the vessel, cells different from the keratinocytes, and
(ii) after the first trypsinization, subjecting the cell population having adhered to the vessel to second trypsinization for detaching the keratinocytes from the vessel.

4. The method as set forth in claim 3, wherein
the cell population including the keratinocytes is exposed to trypsin for not less than one minute and not more than three minutes in the first trypsinization, and
the cell population from which the cells detached by the first trypsinization have been removed is exposed to trypsin for not less than three minutes in the second trypsinization.

5. A method for producing keratinocytes, comprising a culture step of serially culturing, in the presence of feeder cells, an Rho kinase inhibitor, and an extracellular matrix, keratinocytes induced from pluripotent stem cells, wherein
in the culture step, a separation step of subjecting a cell population including the keratinocytes induced from pluripotent stem cells to trypsinization so as to separate the keratinocytes is carried out a plurality of times, and
the separated keratinocytes are serially cultured in the presence of feeder cells, an Rho kinase inhibitor, and an extracellular matrix.

6. The method as set forth in claim 5, wherein the Rho kinase inhibitor is Y27632.

7. The method as set forth in claim 5, wherein the extracellular matrix is laminin or a fragment thereof.

8. (canceled)

9. (canceled)

10. Keratinocytes induced from pluripotent stem cells and having a cell population doubling level (PDL) of more than 15.

11. The keratinocytes as set forth in claim 10, wherein the keratinocytes have a cell population doubling time of not more than 60 hours.
